(19) United States
(12) Patent Application Publication
(10) Pub. No.: US 2008/0027526 A1
(43) Pub. Date: Jan. 31, 2008

(54) LEAD COMPRISING A DRUG REGION SHARED BY MORE THAN ONE ELECTRODE

(75) Inventor: Paul E. Zarembo, Vadnais Heights, MN (US)

Correspondence Address: SCHWEGMAN, LUNDBERG & WOESSNER, P.A., P.O. BOX 2938, MINNEAPOLIS, MN, 55402, US

(73) Assignee: CARDIC PACEMAKERS, INC., ST. PAUL, MN (US)

(21) Appl. No.: 11/460,429

(22) Filed: Jul. 27, 2006

(51) Int. Cl. A61N 1/05

(52) U.S. Cl. 607/120

(57) ABSTRACT

One or more multi-electrode lead couplable with a medical device, such as an implantable medical device. Each lead includes a lead body extending from a lead proximal end portion to a lead distal end portion. The proximal end portion includes a connector assembly for connection to the medical device. An intermediate or distal end portion includes two or more electrodes and a drug region shared by at least two of the electrodes. In one example, the drug region is positioned between two or more electrodes such each of the electrodes may benefit from a drug in the region. In another example, the medical device comprises circuitry adapted to sense a heart in a first instance and stimulate the heart in a second instance using a selected electrode configuration. A method of forming a lead having a drug region shared by more than one electrode is also discussed.

### TECHNICAL FIELD

This patent document pertains generally to leads for linking medical devices with selected bodily tissue to be sensed or stimulated by such devices. More particularly, but not by way of limitation, this patent document pertains to a lead comprising a drug region shared by more than one electrode and systems and methods related thereto.

### BACKGROUND

Leads represent the electrical link between a medical device, such as an implantable medical device (referred to as “IMD”), and a subject's cardiac or other bodily tissue, which is to be sensed or stimulated. A lead generally includes a lead body that contains one or more electrical conductors extending from a proximal end portion of the lead to an intermediate or distal end portion of the lead. The lead body includes insulating material for covering and electrically insulating the electrical conductors. The proximal end of the lead further includes an electrical connector assembly couplable with the IMD, while the intermediate or distal end portion of the lead includes one or more tissue sensing/stimulation electrodes that may be placed within, on, or near a desired sensing or stimulation site within the body of the subject.

The safety, efficacy, and longevity of an IMD depend, in part, on the performance and properties of the lead(s) used in conjunction with the device. For example, various properties of a lead and the one or more electrodes thereon will result in a characteristic lead impedance and stimulation threshold. Lead impedance corresponds to an electrical resistance of a lead to direct current. Stimulation threshold is the energy required in a stimulation pulse to depolarize, or “capture,” the cardiac or other bodily tissue to which a pulse is directed. A relatively low threshold and impedance is desirable to minimize the current drawn from a battery of the IMD in delivering a stimulation pulse. Maximizing the useful life of the battery is important to extend the useful life of the IMD, thereby reducing the need to replace the implanted device.

One factor that can affect the stimulation threshold, particularly during the first several weeks after implantation of a lead, is the natural immunological response of the subject's body to the lead as a foreign object. The presence of the lead activates macrophages, which attach themselves to the surface of the lead and any electrodes thereon and form multi-nucleated giant cells. These cells, in turn, secrete various substances, such as hydrogen peroxide as well as various enzymes, in an effort to dissolve the foreign object. Such substances, while intending to dissolve the foreign object, also inflict damage to the surrounding tissue. When the surrounding tissue is the myocardium, these substances cause necrosis. Areas of necrosis, in turn, impair the electrical characteristics of the electrode-tissue interface. Consequently, stimulation thresholds may rise.

Even after the microscopic areas of tissue die, the inflammatory response continues and approximately seven days after implant, the multi-nucleated giant cells cause fibroblasts to begin laying down collagen to replace the necrotic myocardium. Eventually, on the order of three weeks or so after implant, the lead and its associated electrodes are encapsulated by a thick layer of fibrotic tissue. Typically, the inflammatory response ends at this time. The fibrotic encapsulation of the lead and its tissue electrodes, however, remains. Since the fibrotic tissue is not excitable tissue, an elevated stimulation threshold can persist due to the degraded electrical properties of the electrode-tissue interface.

Another factor that can affect the stimulation and impedance thresholds pertain to the location of electrodes relative to the subject's cardiac or other bodily tissue to be sensed or stimulation, and in this way, pertains to the limited number of electrodes that a typical lead possesses. An electrode's ability to sense or stimulate the subject's cardiac or other bodily tissue depends, in part, on the relative location of the electrode(s) within, on, or near such tissue and the interface therebetween. Typically, the distal or intermediate portion of the lead body includes one or two electrodes arranged in a unipolar or bipolar arrangement. A unipolar arrangement includes one tissue electrode, which represents one pole of an electrical circuit, while the other pole is represented by the body of the IMD itself. A bipolar arrangement includes a pair of tissue electrodes that form the single electrical circuit (i.e., one electrode is positive, while the other electrode is negative). Through the use of leads having only one or two tissue electrodes, the sensing or stimulation is limited, sometimes to a tissue location different from the optimum or acceptable position (e.g., a position having a lower stimulation and impedance parameter). Sensing or stimulating at such undesirable locations results in greater IMD battery drain, and thus, reduced IMD life.

### SUMMARY

A lead comprises a lead body extending from a lead proximal end portion to a lead distal end portion, and having an intermediate portion therebetween. An electrical connector assembly is coupled to the lead proximal end portion, while at least a first and a second electrode are disposed along the lead intermediate or distal end portion. The first and second electrodes are electrically coupled to the connector assembly by way of one or more longitudinally extending conductors. A drug region is disposed between the first and second electrodes, such that a drug in the region may be shared by the electrodes.

Several options for the lead are as follows. In one example, the drug region comprises a polymeric material mixed with a drug. In another example, the drug region comprises a drug eluting matrix that elutes one or more drugs over time. In one such example, the drug eluting matrix comprises at least one drug and at least one drawing agent. The drawing agent has the ability to draw bodily fluid into the matrix for modulating a drug delivery rate of the at least one drug to nearby bodily tissue. In a further example, the lead body comprises a preformed biased portion, such as a helical or sinusoidal curve shape, at one or both of the lead intermediate or lead distal end portion.

A cardiac system includes a lead and a medical device, such as an IMD. The lead includes at least two electrodes and a shared drug region disposed near the at least two electrodes. In one such example, the lead includes four electrodes and two shared drug regions. The medical device includes an electronics circuit configured to generate one or both of a sense signal or a stimulation signal, which are delivered using one or more of the lead electrodes. According to at least one example, a processing circuit of the medical device is adapted to select the delivering electrode(s) using, at least in part, one or a combination of a stimulation threshold parameter, a stimulation impedance parameter, a stimulation selection parameter, a heart chamber configuration parameter, or a spatial distance parameter.

An implantable lead includes a lead body extending form a proximal end portion to a distal end portion, and having an intermediate portion therebetween. The lead body includes at least one elongated electrical conductor contained therewithin. Two or more electrodes are disposed on the lead body and electrically coupled with the at least one conductor. A drug region is positioned and configured to dispense a drug adjacent the two or more electrodes.

Several options for the implantable lead are as follows. In one example, the drug region is positioned between the two or more electrodes. In another example, a structural strength or fixation mechanism is disposed on the lead body near an edge of the drug region. In yet another example, the two or more electrodes are electrically coupled to one another to provide an increased (effective) electrode sense surface area. In one such example, the electrodes are electrically coupled using a hard (wire) connection therebetween. In another such example, the electrodes are electrically coupled using a programmed software connection with an attached medical device.

A method of manufacturing a lead comprises forming a lead body encasing a substantial portion of one or more electrical conductors, including forming a lead body extending from a proximal end to a distal end and having an intermediate portion therebetween. The method further comprises disposing a first and a second electrode on the lead body, such that the electrodes are separated a select distance away from one another. Further yet, the method comprises disposing a drug region on the lead body in a position such that the drug is shared by the first and second electrodes.

Several options for the method are as follows. In one example, disposing the drug region on the lead body includes spraying, dipping, or painting the drug on the lead body. In another example, disposing the drug region on the lead body includes fusing a drug ring to the lead body. In yet another example, forming the lead body includes forming a bias portion at or near the lead intermediate or distal end portion. Additionally, the method may further include electrically coupling the first and second electrodes, or disposing a third and fourth electrodes and an associated drug region on the lead body.

The leads, systems, and methods discussed herein may overcome many deficiencies of current leads, systems, and methods. As one example, through the use of a lead including a drug region shared by more than one electrode, less drug may be used on a per lead basis in comparison to conventional leads in which a separate drug region is associated with each individual electrode (for which a drug and its associated benefits is desired). As another example, through the use of the drug shared region, additional regulatory approval may not be needed for a lead including three, four or more electrodes, as testing has already been conducted for leads including two drug regions. For instance, a lead including four electrodes and two drug regions shared by the four electrodes (e.g., a first and second electrode sharing a first drug region and a second and third electrode sharing a second drug region) need not require additional drug safety and efficacy testing, as such testing has already been performed for leads having two electrodes and two associated drug regions.

As yet another example, through the use of a lead including three, four or more electrodes, the opportunity exists for a caregiver (e.g., a physician) or an IMD itself to choose among numerous electrode configurations for sensing or stimulating the desired cardiac or other bodily tissue. The numerous possible electrode configurations allow the caregiver or the IMD to recurrently select one or more electrode configurations, which optimize or provide an acceptable balance of, among other things, one or a combination of a stimulation threshold parameter, a stimulation impedance parameter, a stimulation selection parameter (including reduction of phrenic nerve or diaphragmatic stimulation), a heart chamber configuration parameter, or a spatial distance parameter.

These and other examples, advantages, and features of the present leads, systems, and methods will be set forth, in part, in the detailed description that follows, and in part, will become apparent to those skilled in the art by reference to the following description and drawings or by practice of the same.

### BRIEF DESCRIPTION OF THE DRAWINGS

In the drawings, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic view of an implantable cardiac system and an environment in which the system may be used, as constructed in accordance with at least one embodiment.

FIG. 2 is an enlarged schematic view of the implantable cardiac system of FIG. 1, as constructed in accordance with at least one embodiment.

FIG. 3 is a schematic view of an implantable neurological system and an environment in which the system may be used, as constructed in accordance with at least one embodiment.

FIGS. 4A-4I are plan views of an intermediate or distal portion of a lead, each constructed in accordance with at least one embodiment.

FIG. 5 is a plan view of a lead, as constructed in accordance with at least one embodiment.

FIG. 6A-6B are plan views of a lead, each constructed in accordance with at least one embodiment.

FIG. 7 is a cross-section view of a lead taken along a line, such as line 7-7 of FIG. 4A, as constructed in accordance with at least one embodiment.

FIG. 8 is a schematic view illustrating portions of an implantable system, including circuitry of an IMD, as constructed in accordance with at least one embodiment.

FIG. 9 is a flow diagram illustrating a method of making a lead, as performed in accordance with at least one embodiment.

### DETAILED DESCRIPTION

The following detailed description includes references to the accompanying drawings, which form a part of the detailed description. The drawings show, by way of illustration, specific embodiments in which the present leads, systems, and methods may be practiced. These embodiments, which are also referred to herein as “examples,” are described in enough detail to enable those skilled in the art to practice the present leads, systems, and methods. The embodiments may be combined, other embodiments may be utilized or structural, logical, and electrical changes may be made without departing from the scope of the present leads, systems, and methods. It is also to be understood that the various embodiments of the present leads, systems, and methods, although different, are not necessarily mutually exclusive. For example, a particular feature, structure or characteristic described in one embodiment may be included within other embodiments. The following detailed description is, therefore, not to be taken in a limiting sense, and the scope of the present leads, systems, and methods are defined by the appended claims and their legal equivalents.

In this document the terms “a” or “an” are used to include one or more than one; the term “or” is used to refer to a nonexclusive or, unless otherwise indicated; the term “subject” is used synonymously with the term “patient”; and the terms “implantable medical device,” “implantable lead,” and the like refer to elements that are to be at least partially placed within a subject's body for a period of time for which it would be beneficial to have a drug region present. In addition, it is to be understood that the phraseology or terminology employed herein, and not otherwise defined, is for the purpose of description only and not of limitation.

Leads, systems, and methods are provided herein for, among other things, minimizing an amount of drug needed on a per lead basis and minimizing new drug safety and efficacy testing, while still providing multiple vectors and electrode spacing for sensing and stimulation of a subject's bodily tissue. The foregoing is achieved, in part, by positioning two or more electrodes on a lead in such a way that the electrodes can share, thereby reaping the benefits of, a single drug region.

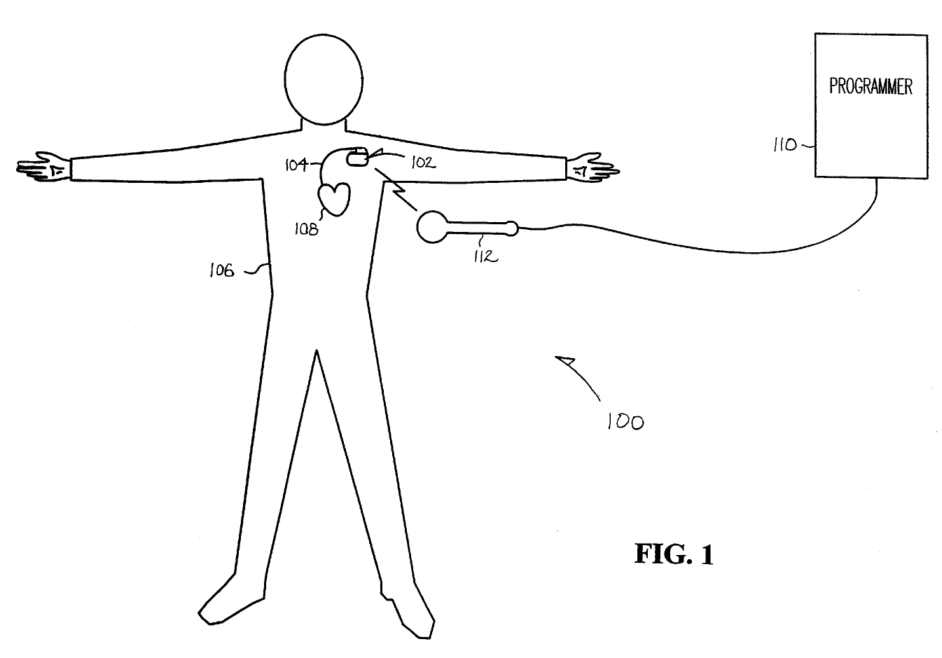

Turning now to the drawings, and initially to FIG. 1, which illustrates an implantable cardiac system 100 and an environment (e.g., a subcutaneous pocket made in the wall of a subject's 106 chest, abdomen, or elsewhere) in which the system may be used. In varying examples, the cardiac system 100 may be used for receiving or delivering electrical signals or pulses to sense or stimulate, respectively, a heart 108 of the subject 106. As shown in FIG. 1, the cardiac system 100 may include an IMD 102, at least one implantable lead 104 coupled with the IMD on a proximal end, and an external programmer 110 adapted to electrically communicate with the IMD, such as wirelessly through the use of a telemetry device 112.

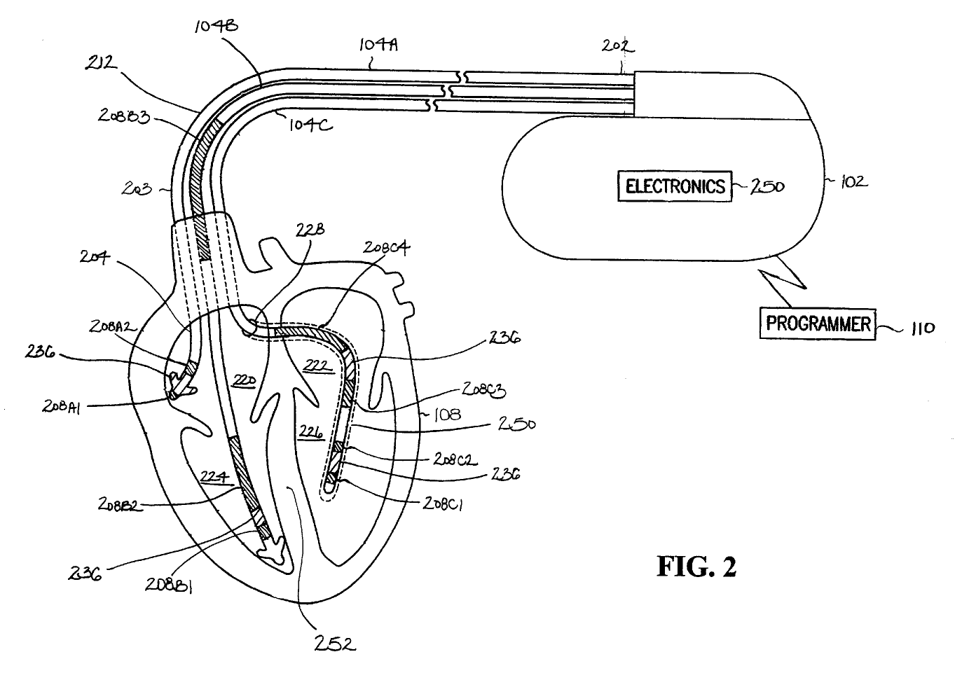

The IMD 102 generically represents, but is not limited to, cardiac function management (referred to as “CFM”) systems such as pacemakers (also referred to as “pacers”), cardioverters/defibrillators, pacers/defibrillators, biventricular or other multi-site resynchronization or coordination devices such as cardiac resynchronization therapy (referred to as “CRT”) devices, sensing instruments, or drug delivery systems. Among other things, the IMD 102 includes a source of power as well as an electronics circuitry portion 250 (see, e.g., FIG. 2). In varying examples, a housing of the IMD 102 may serve as an indifferent electrode for use in combination with an electrode disposed on the lead 104 (see, e.g., FIG. 2).

In one example, the IMD 102 is a pacemaker. Pacemakers deliver timed sequences of low energy electrical stimuli, called pace pulses, to the heart 108, such as via the at least one lead 104 having one or more (typically ring-like) electrodes disposed within, on, or near the heart. Heart 108 contractions are initiated in response to such pace pulses (i.e., the pulses capture the heart 108). By properly timing the delivery of pace pulses, the heart 108 can be induced to contract in proper rhythm, greatly improving its efficiency as a pump. Pacemakers are often used to treat subjects 106 with bradyarrhythmias, that is, hearts 108 which beat too slowly or irregularly. Pacemakers may also coordinate atrial and ventricular contractions to improve a heart's 108 pumping efficiency.

In another example, the IMD 102 is a CRT device for coordinating the spatial nature of heart depolarizations for improving a heart's pumping efficiency, such as for subjects 106 experiencing CHF. In one such example, the CRT device may deliver appropriate timed pace pulses to different locations of the same heart 108 chamber to better coordinate the contraction of that heart chamber, or the CRT device may deliver appropriately timed pace pulses to different heart 108 chambers to improve the manner in which these different heart chambers contract together, such as to synchronize left and right side contractions.

In yet another example, the IMD 102 is a defibrillator that is capable of delivering higher energy electrical stimuli to the heart 108 (as compared to, for example, pacing pulses). Defibrillators may include cardioverters, which synchronize the delivery of such stimuli to sensed intrinsic heart activity signals. Defibrillators are often used to treat subjects with tachyarrhythmias, that is, hearts which beat too quickly. Such too-fast heart rhythms cause diminished blood circulation because the heart isn't allowed sufficient time to fill with blood before contracting to expel the blood. Such pumping by the heart 108 is inefficient. A defibrillator is capable of delivering a high energy electrical stimulus via a (typically coil-like) electrode that is sometimes referred to as a defibrillation countershock, also referred to simply as a “shock.” The countershock interrupts the tachyarrhythmia, allowing the heart 108 to reestablish a normal rhythm for the efficient pumping of blood.

When the IMD 102 is a defibrillator, it may also be used to treat subjects experiencing cardiac arrest in which the heart 108 stops beating or goes into fibrillation (i.e., inefficient pumping). The high energy defibrillation countershocks deliverable by a defibrillator may restart the heart 108 or stop fibrillation thereby allowing the heart 108 to re-establish normal sinus rhythm.

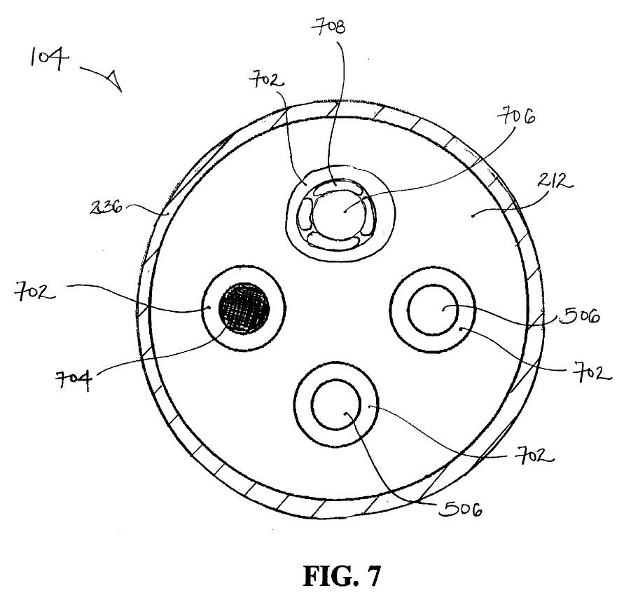

FIG. 2 illustrates an enlarged schematic view of the cardiac system 100 shown in FIG. 1, for receiving and delivering electrical signals (e.g., via an electronics circuitry portion 250) to sense or stimulate a subject's heart 108, which includes a right atrium 220, a left atrium 222, a right ventricle 224, a left ventricle 226, a coronary sinus 228 extending from the right atrium, and a coronary vein 250. The cardiac system 100 includes a medical device, such as an IMD 102, and at least one lead 104A, 104B, 104C, where each lead extends from a proximal end portion 202 to a distal end portion 204, and has an intermediate portion 203 therebetween. The lead proximal end portion 202 includes an electrical connector assembly to connect to the IMD 102, while the lead intermediate 203 or distal end portion 204 includes two or more electrodes 208. Each lead includes a lead body 212 which, in one example, is comprised of a tubing material formed of a biocompatible polymer suitable for implementation within a subject's body 106 (FIG. 1), such as silicone rubber. The lead body 212 of each lead includes at least one lumen (see, e.g., FIG. 7) which house longitudinally electrical conductors extending from the connector assembly to the tissue sensing/stimulation electrodes. The electrical conductors carry current and other signals between the IMD 102 and the electrodes 208.

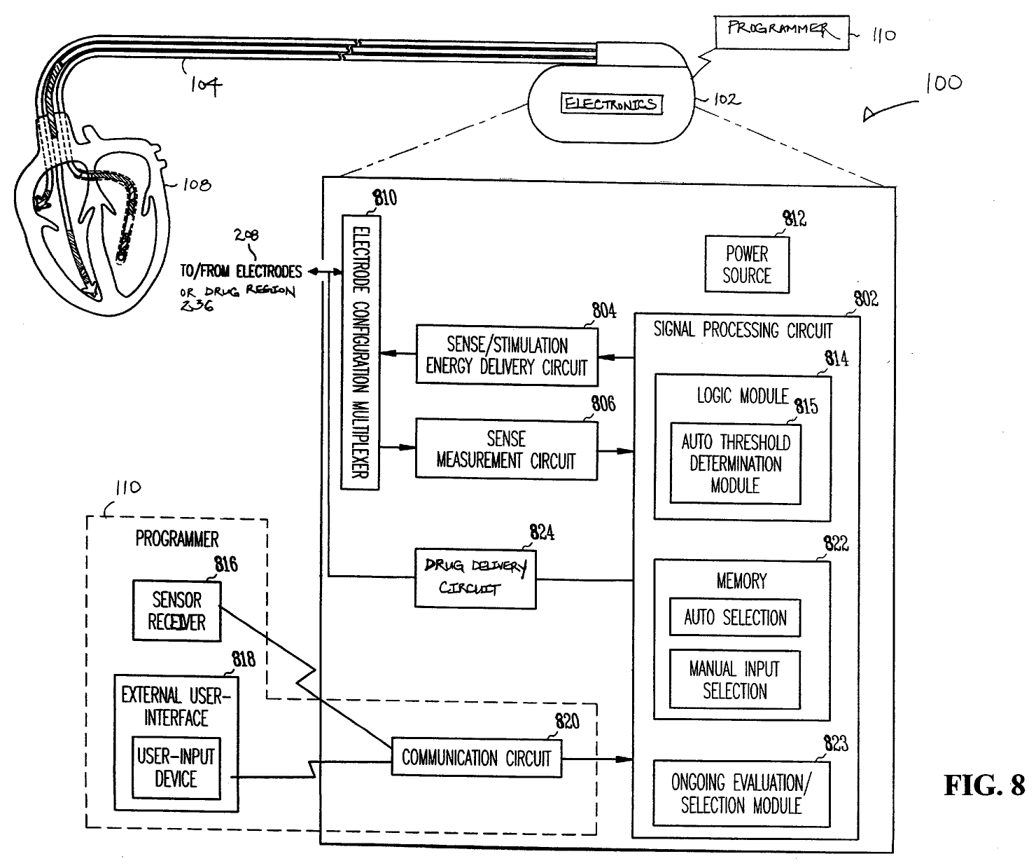

In this example, the atrial lead 104A includes electrodes disposed in, around, or near the right atrium 220 of the heart, such as ring electrode 208A2 and tip electrode 208A1, for sensing signals (e.g., via a sense measurement circuit 806 (FIG. 8)) or delivering pacing therapy (e.g., via a stimulation energy delivery circuit 804 (FIG. 8)) to the right atrium. Atrial lead 104A may also include additional electrodes, such as for delivering atrial or ventricular cardioversion/defibrillation or pacing therapy to the heart 108. Also shown in this example, a right ventricular lead 104B includes one or more electrodes, such as a ring electrode 208B1, for sensing signals or delivering pacing therapy. The right ventricular lead 104B may also include additional electrodes, such as coil electrodes 208B2 or 208B3 for delivering right atrial or right ventricular cardioversion/defibrillation or pacing therapy to the heart 108. As further shown in this example, the system 100 may also include a left ventricular lead 104C, which provides one or more electrodes such as tip electrode 208C1 and ring electrode 208C2, for sensing signals or delivering pacing therapy. The left ventricular lead 104C may also include one or more additional electrodes, such as coil electrodes 208C3 or 208C4 for delivering left atrial or left ventricular cardioversion/defibrillation or pacing therapy to the heart 108.

Although not shown in FIG. 2, other dispositions of the lead intermediate 203 and distal end 204 portions within, on, or near the heart 108 are also possible. For instance, in one example a lead 104 includes at least one preformed biased portion or is otherwise configured to urge one or more of the electrodes 208 thereon against a septal wall 252 for pacing the of the same, such as is discussed in commonly assigned Hansen, U.S. patent application Ser. No. 11/230,989, entitled “MULTI-SITE LEAD/SYSTEM USING A MULTI-POLE CONNECTION AND METHODS THEREFOR,” filed on Sep. 20, 2005 (Attorney Docket No. 279.822US1), which is hereby incorporated by reference in its entirety.

Disposed between the electrode pairs 208A1-208A2, 208B1-208B2, 208C1-208C2, and 208C3-208C4 is a drug region 236, which may be shared by each electrode of the associated electrode pair. The incorporation of a shared drug region in a lead may provide for, among other things, lower lead impedances (as optimal electrode vectors with adjacent drug regions may be chosen), or lower peak and chronic stimulation thresholds by reducing, for example, inflammation or fibrotic growth. A reduction in lead impedance and stimulation thresholds increases the longevity of medical devices, such as the IMD 102, because the current drain from the IMD's power source is reduced. In addition, a lead construction in which two or more electrodes share a drug region, such as a drug collar, advantageously minimizes an amount of drug needed on a per lead basis—resulting in a cost savings—and further minimizes new drug safety and efficacy testing which would be required for leads having more than two drug regions (there may be instances in which more drug would potentially be detrimental).

Previously tested leads comprised a separate drug region for each electrode for which an adjacent drug region and its associated benefits was desired. For instance, a lead including two electrodes would typically include two associated drug regions. As mentioned above, by having only two or less electrodes per lead limited sensing and stimulation to a limited number of electrode configurations. With the advent of quad-polar leads (i.e., leads having four electrodes, which may find utility in treating congestive heart failure by allowing switching of pacing electrodes) and the like, the question may arise as to what the proper dosage of drug per lead should be, and further, is it acceptable to include one drug ring per electrode. Placing an electrode on each side of a drug region eliminates the need to resolve the dosage question and thus, may eliminate any potential need for new clinical studies (e.g., by regulatory agencies, such as the Food and Drug Administration (FDA) or British Standards Institution (BSI)) as the dosage is the same as historical data.

Also shown in FIG. 2 is a programmer 110. In this example, the programmer is an external-type programmer that may be used to program many of the parameters of the electronics circuitry portion 250 or other parameters of the IMD 102. In another example, the programmer 110 may be an external handheld-type programmer adapted for use by a subject 106. Another type of programmer 110 might be one that a physician would have in his/her office, which can be used to program various parameters associated with, for example, stimulation signals produced by the IMD 102. The programmer 110 may includes a feature allowing for a readout of the status of the IMD 102.

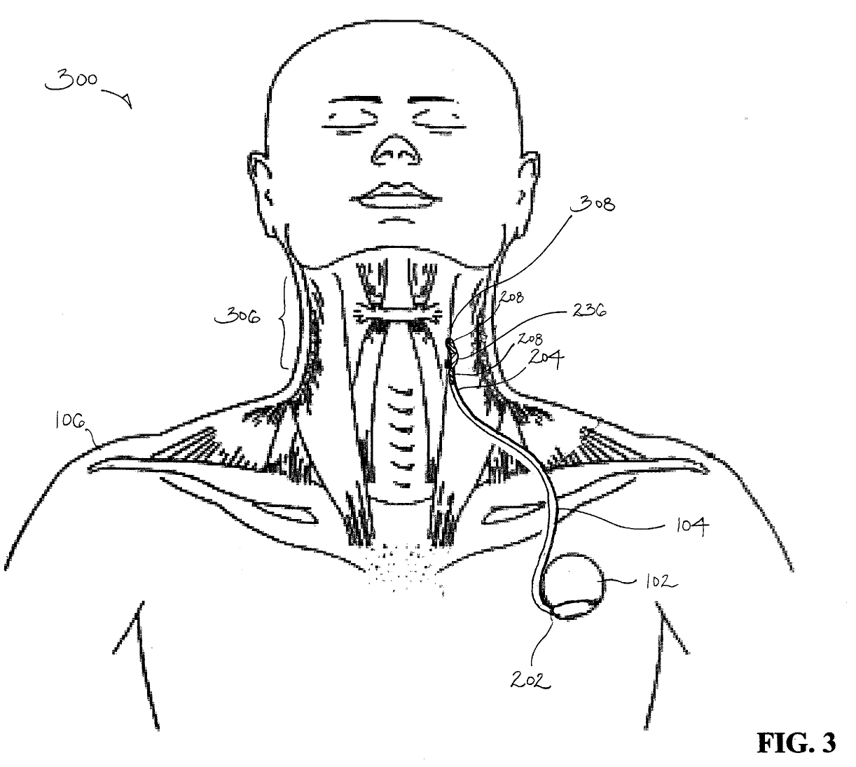

The present leads, systems, and methods may be used in a wide variety of medical applications including, but not limited to, cardiac pacing, defibrillation, cardioversion, or as shown in FIG. 3, neurostimulation. FIG. 3 illustrates an exemplary neurostimulator system 300 implanted in a subject 106. The system 300 comprises an IMD 102 and a lead 104 extending from a lead proximal end portion 202 coupled with the IMD to a lead distal end portion 204. The lead includes at least two electrodes 208 and a drug region 236 shared by the at least two electrodes. The IMD may be implanted subcutaneously in the subject 106, such as in the abdomen or chest region. From the location of implantation of the IMD 102, the lead distal end portion 204 is tunneled subcutaneously to the subject's neck region 306 and positioned in proximity to a desired therapy site. In the example of FIG. 3, the target therapy site is the vagal nerve 308. In another example, the target therapy site is one or more baroreceptor in a pulmonary artery. The lead 104 is positioned such that the at least two electrodes 208 and the drug region 236 are in close proximity to the desired therapy site.

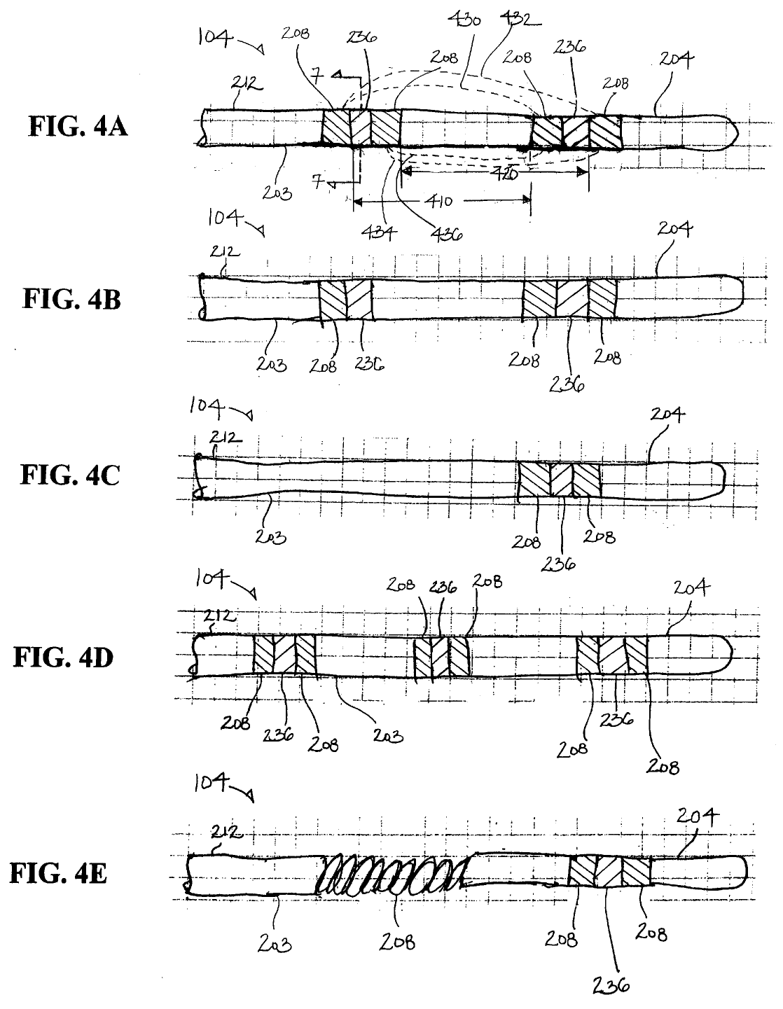

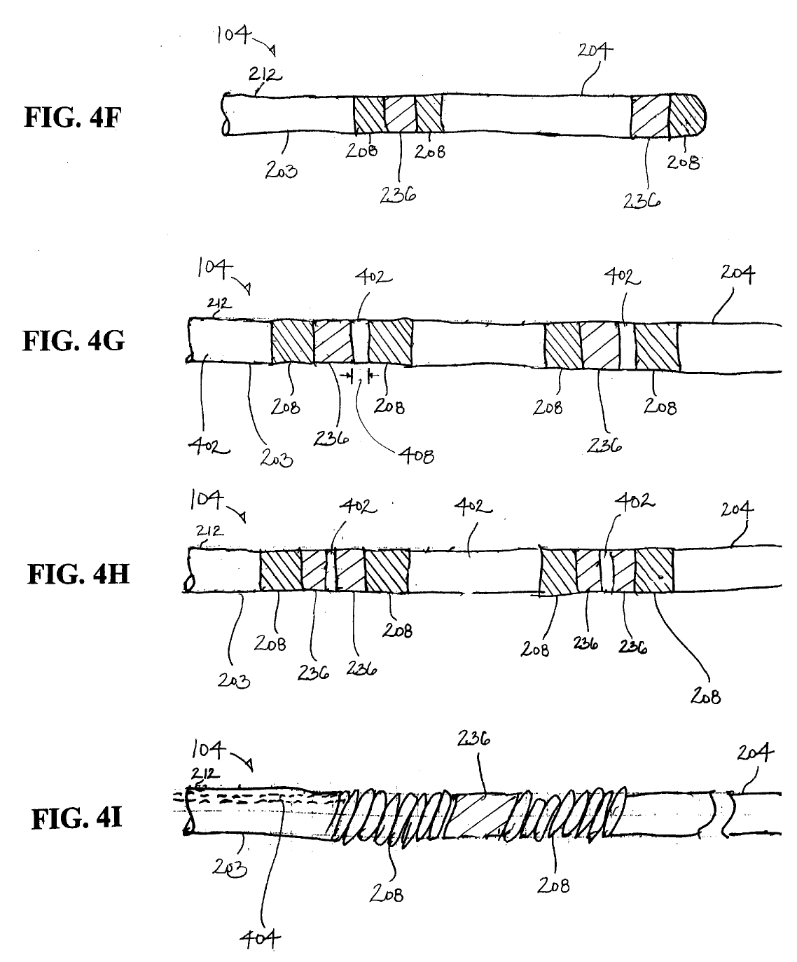

FIGS. 4A-4I illustrate various examples of a lead intermediate 203 or distal end 204 portion according to the present subject matter. Each lead intermediate 203 or distal end portion 204 extends from a proximal end portion 202 (FIG. 2), which includes an electrical connector assembly adapted to couple to a medical device, such as an IMD 102 (FIG. 1). The IMD contains an electronics circuitry portion 250 (FIG. 2) and software necessary to detect, for example, certain types of arrhythmias and to correct for them. Each lead 104 also includes a lead body 212 to which two or more electrodes 208 and a shared drug region 236 are disposed. In varying examples, the drug region 236, such as a drug collar, is shared by placing at least one electrode 208 adjacent or near each side of the drug region.

As discussed above, the implantation of a lead 104 into a subject's 106 body may, among other things, vitiate a stimulation pulse's desired effects. For example, reactions between the body and lead materials may encourage fibroses. In regards to pacing (i.e., one form of stimulation), fibrosis is considered a factor in the increase in chronic stimulation threshold, and thus increased device battery drain, that may be experienced over time. Also, the mechanical trauma of implantation can result in inflammation of the adjacent bodily tissue. This inflammation can further alter the response of the tissue to the pacing stimulus, both acutely and chronically. Other interactions between the lead 104 and body, while not directly affecting the tissue's response to stimulation, are nonetheless undesirable. In some circumstances, the body region to be stimulated may be irritable. The implantation of a lead 104 can compound this irritability. For example, the presence of an implanted lead 104 can promote thrombus formation. For at least these reasons, the present leads 104 comprise a drug region 236 shared by the at least two electrodes 208.

In varying examples, the drug region(s) 236 is positioned between the at least two electrodes 208. The drug region releases a selected drug, such as a steroid, adjacent to the point of sensing or stimulation. The selected drug or combination of drugs from the drug region is used to avoid acute and chronic increases in the stimulation threshold caused by inflammation or fibrosis, for example. In addition, thrombus formation may generally be avoided or reduced by the administration of suitable drugs. Regardless of each drug's purpose, a threshold dose of the drug must be provided in order to evoke a desired effect. Advantageously, the present leads include a drug region 236 configured and positioned to deliver (e.g., elude) the requisite amount of drug needed to come into contact with a desired electrode or to effectuate a desired outcome for actions of the at least two electrodes 208.

In particular, FIG. 4A illustrates a lead 104 having four electrodes 208 and two drug regions 236. The drug regions 236 are disposed such that one region is between a first and second electrode (i.e., a first electrode pair), while the other drug region 236 is between a third and a fourth electrode (i.e., a second electrode pair). The electrodes comprising each electrode pair can be positioned close together on the lead body 212 to accommodate a (relatively) short drug region 236, or they can be spaced far apart for use with a (relatively) long drug region 236. Even if the first and second or third and fourth electrodes are positioned close together on the lead body 212, the distance between the first and third electrodes 410 or the second and fourth electrodes 420 can be set to a desired sensing or stimulation distance (e.g., 10-11 mm).

As shown in FIG. 4A, the shared drug region (lead) construction still allows for multiple sensing or stimulation vectors, such as 430, 432, 434, 436, among others. As discussed in commonly assigned Hansen, U.S. patent application Ser. No. 11/230,989, entitled “MULTI-SITE LEAD/SYSTEM USING A MULTI-POLE CONNECTION AND METHODS THEREFOR,” filed on Sep. 20, 2005, which is hereby incorporated by reference in its entirety, multiple sensing or stimulation vectors advantageously allow for an electrode configuration which provides a desirable combination of electrode contact with myocardial tissue, low stimulation thresholds, avoidance of unintended stimulation of the phrenic nerve or diaphragm, or beneficial heart remodeling. In addition, the first and second or third and fourth electrodes may be electrically coupled (e.g., a hard (wire) connection or in the IMD 102 electronically), thereby providing an increased surface area (and lower thresholds) to sense or stimulate from.

As illustrated in FIGS. 4B-4I, the present subject matter is not limited to a lead 104 having four electrodes (i.e., a quad-polar lead) and two drug sharing regions 236; rather, any number, type (e.g., ring-like or coil-like), or combination of electrodes 208 and drug regions 236 may be used, such that two or more of the electrodes share the same drug region 236.

Referring specifically to FIGS. 4G-4H, additional features 402 may be placed near an edge of a drug region 236 to provide structural strength or fixation mechanisms to the lead 104. For instance, polyurethane rings may be placed adjacent to the one or more drug regions 236 to increase axial strength of the lead 104. Such polyurethane rings may be fused or bonded to the underlying lead material, for example. As further shown in FIGS. 4G-4H, the shared drug region 236 need not be directly adjacent the two or more electrodes 208 to which it is shared. Rather, the shared drug region 236 may be positioned a short distance 408 (e.g., between about 0 to about 0.250″)away from one or both of the two or more electrodes 208 so long as it can provide an anti-inflammatory or other benefit to the electrodes at desired times.

As shown in FIG. 41, the two or more electrodes 208 that share a drug region 236 may comprise coil electrodes. Typically, it is the (ring-like) pacing electrodes (as shown in FIG. 4H) which have the most need for a drug region positioned nearby; however, coil-like electrodes (as shown in FIG. 41) may also have a need for a shared drug region in certain circumstances such as for reducing inflammation due to electrode abrasion on tissue or for reducing inflammation after trauma of shock. Some drug formulations may lower shocking thresholds. In one example, a biocompatible cable 404 (e.g., Pt-Clad Tantalum) goes from a straight cable to a wound coil electrode. This wound coil electrode could be made longer, as needed, to better ensure contact with myocardium or other bodily tissue.

Contents, structure, and size of the shared drug region 236 may vary depending on, among other things, the desired use of the region. As one example, the drug comprised in the drug region 236 may be one which is intended to counter thrombus formation, fibrosis, inflammation or arrhythmias, or any combination of drugs intended to accomplish one or more of these purposes, or any drug or combination of drugs intended to accomplish any other desirable localized purpose or purposes. As another example, the drug region 236 may be of any length or thickness to contain and apply the desired amount of drug to each electrode to which it is shared. As yet another example, the drug region 236 may be a separate element (e.g., a collar-like structure) secured to the lead body 212 (FIG. 2) or may be integrally molded into the lead body.

In one specified example, the drug region 236 comprises a carrier material and a drug. Typically, the carrier material is selected and formulated for an ability to incorporate the desired drug during manufacture and release the drug within a subject 106 (FIG. 1) after implantation. The carrier material may comprise, among other things, silicone rubber or other polymer (e.g., polyurethane, polyethylene, ethylene-tetrafluoroethylene (ETFE), polytetrafluoroethylene (PTFE), polyetheretherketone (PEEK)) or material (e.g., metal, porous ceramics) that can hold or elute a drug. Alternatively, the carrier material may comprise a porous or non-porous material onto which a drug may be collated. The amount of any particular drug incorporated into the drug region 236 is often determined by the effect desired, the drug's potency, or the rate at which the drug capacity is released from the carrier material, as well as other factors.

In another specified example, the drug region 236 comprises a drug eluting matrix that elutes over time. In one such example, the drug eluting matrix is a steroid compounded with an uncured silicone rubber. Upon curing, the steroid becomes incorporated into a hardened polymeric binder. The curing process, in one example, is performed within a mold to produce a desired matrix shape. For instance, for a pacing lead, a rod or tube of dexamethasone acetate in silicone rubber is cut to form a plug or ring, respectively.

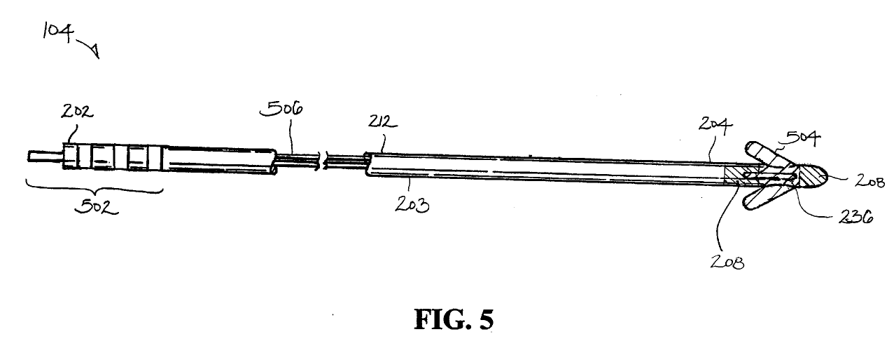

FIG. 5 illustrates a lead 104 having a lead body 212 extending from a lead proximal end portion 202 to a lead distal end portion 204 and having a lead intermediate portion therebetween 203. The lead proximal end portion 202 includes a connector assembly 502 adapted to couple with a medical device, such as an IMD 102 (FIG. 1), and specifically an electronics circuitry portion 250 contained within the IMD (FIG. 2). The lead distal end portion 204 includes one electrode 208 proximal and distal to a tine region 504, used for fixing the lead 104 at a desired location within a subject 106 (FIG. 1). The electrodes 208 are coupled with the connector assembly 502 via one or more electrical conductors 506 contained within the lead body 212. FIG. 5 further illustrates that a drug region 236 may be disposed on, or integrated with, portions of the tine region 504 and be shared by each of the electrodes 208.

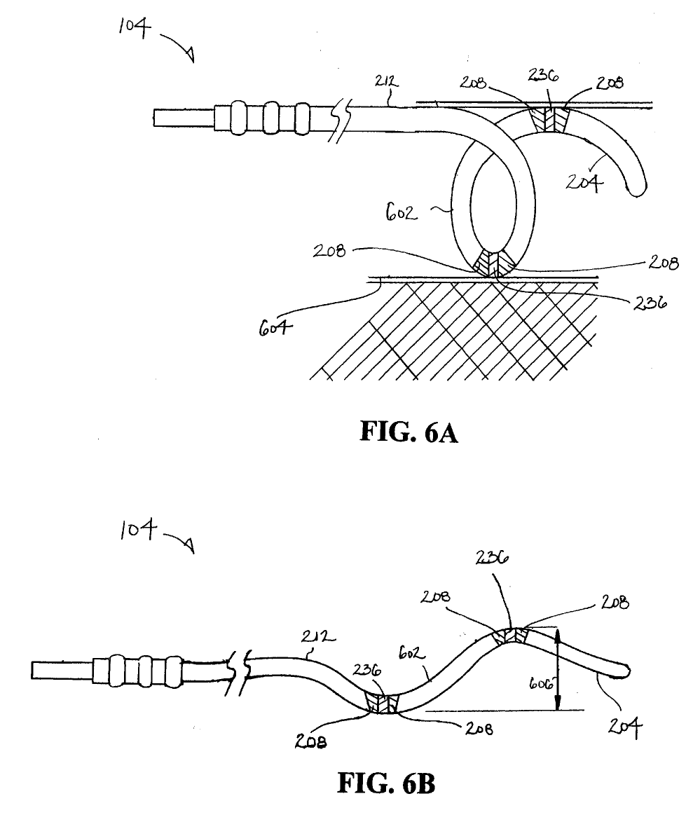

As discussed above, it is advantageous that an electrode configuration used to stimulate bodily tissue have a low stimulation threshold to reduce device battery drain, and thus, increase device life, or eliminate phrenic nerve or diaphragmatic stimulation. FIGS. 6A-6B illustrate leads 104 having a preformed bias portion 602 at a lead intermediate 203 or distal end portion 204. The preformed bias portions 602 may help ensure a reliable and stable lead/vessel wall area interface, and in turn, lower stimulation thresholds. Specifically, FIG. 6A illustrates a lead 104 having a helical preformed bias portion 602, while FIG. 6B illustrates a lead 104 having a sinusoidal curve preformed bias portion 602. The leads 104 including the preformed bias portion 602 may include two or more electrodes 208 positioned on the lead body 212 to share a drug region 236. As shown in FIG. 6A, the electrodes 208 and shared drug region 236 may be positioned on the preformed bias portion 602 to contact a vessel (e.g., coronary vein 250) wall 604. As shown in FIG. 6B, the preformed bias portion 602 may include a curve height 606 of a variety of sizes.

Leads 104 having the preformed biased portion 602 will typically include a lumen 706 (FIG. 7) into which a stylet or guidewire may be inserted. The stylet or guidewire are typically wires that straighten out the lead 104 while it is being placed within a heart 108 or other desired portion of a subject 106 (FIG. 1). By removing the stylet or guidewire, the lead will take on its natural or preformed shape, which in the example of FIG. 6A is a helical curve and in the example of FIG. 6B is a sinusoidal curve.

FIG. 7 illustrates a cross-sectional view of a lead 104, such as taken along line 7-7 of FIG. 4A. The lead 104 shown in FIG. 4A includes four electrodes 208 and two shared drug regions 236, one of which is shown here in cross-section. The electrodes 208 are electrically coupled to an electronics circuitry portion 250 (FIG. 2) of an IMD 102 via one or more conductors 506 carried by a plurality of lumens 702 within the lead body 212. As the line along which the cross-section of FIG. 7 is distal to at least one electrode 208, one of the plurality of lumens 702 is shown with a plug 704 therein. A coil conductor 708 optionally used includes a lumen 706 to allow passage of a guidewire or stylet therethrough.

Surrounding the lead body 212 is a first drug region 236 shared by the two most proximal electrodes 208 of the lead 104 shown in FIG. 4A. Any means of depositing the drug region 236 on the lead body 212, whether physical or chemical, may be used. In one example, the drug region 236 comprises a drug ring that is fused to the lead body 212. In another example, the drug region 236 comprises a drug impregnated porous medium, such as ceramic metal or polymer. In yet other examples, the drug region 236 is sprayed, dipped, painted, or similarly deposited on an outer surface of the lead body 212.

FIG. 8 is a schematic drawing illustrating portions of a system 100 adapted to sense or stimulate (e.g., pace, defibrillate, or cardiovert) a heart 108 of a subject 106 (FIG. 1) at multiple locations within, on, or near the same. In the example shown, system 100 includes a hermetically sealed medical device, such as an IMD 102, and an external programmer 110. The IMD 102 is connected to the heart 108 by way of at least one lead 104. In varying examples, the at least one lead 104 includes at least two electrodes 208, which share a drug region 236. In one example, the lead 104 includes four electrodes 208 and two shared drug regions 236. Through the use of shared drug regions 236, the opportunity exists to use leads having more than two electrodes, while still providing the desired drug benefits to each electrode—all without requiring additional safety and efficacy testing and the expense associated with incorporating additional drug regions to a lead. Leads having more than the conventional one or two electrodes provide a greater number of electrode configurations to sense or stimulate across. As a result, an electrode configuration which prolongs the life of the IMD 102, or other useful benefit, may be selected.

Among other things, the IMD 102 includes a signal processing circuit 802, a sense/stimulation energy delivery circuit 804, a sense measurement circuit 806, an electrode configuration multiplexer 810, a drug delivery circuit 824, and a power source 812. Among other things, external programmer 110 includes an external/internal sensor receiver 816 and an external user-interface 818 including a user-input device. The external/internal sensor receiver 816 is adapted to receive subject specific information from one or more internal or external sensor(s).

The signal processing circuit 802 is adapted to sense the heart 108 in a first instance and stimulate the heart in a second instance, each of which occur by way of one or more (optimal) electrode configuration selected from the two or more electrodes 208 of each lead 104 (FIG. 2) implanted within the subject 108 (FIG. 1) (including intralead and interlead combinations) and one or more indifferent electrode (e.g., a header or housing electrode of the IMD 102). In one example, the IMD (specifically, the signal processing circuit 802) is adapted (i.e., programmed) to automatically analyze all possible electrode configurations of the system 100 and select the one or more electrode configuration to be used in sensing or stimulating the heart 108. The IMD 102 may be further adapted (e.g., via an ongoing evaluation/selection module 823) to monitor and re-select the one or more electrode configuration as necessary).

In another example, the programmer 110 is adapted (i.e., programmed) to automatically analyze all possible electrode configurations of the system 100 and select the one or more electrode configuration to be used in sensing or stimulating the heart 108. In yet another example, the one or more electrode configuration used to sense or stimulate the heart 108 is selected manually by a caregiver (e.g., an implanting physician), and communicated to the IMD 102 (e.g., signal processing circuit 802) using a telemetry device 112 (FIG. 1) and a communication circuit 820 of the IMD. In the example shown, such automatic or manual selection of the one or more electrode configuration is stored in a memory 822. In yet another example, the one or more electrode configuration used to sense the heart 108 in a first instance and stimulate the heart in a second instance are the same. In a further example, the one or more electrode configuration used to sense the heart in a first instance and stimulate the heart in a second instance are different.

The one or more electrode configuration may be selected (either automatically or manually) using, at least in part, one or a combination of a stimulation threshold parameter, a stimulation impedance parameter, a stimulation selection parameter, a heart chamber configuration parameter, or a spatial distance parameter, all of which are further discussed below. Other parameters that may be used to select the one or more electrode configuration are discussed in commonly assigned Hansen, U.S. patent application Ser. No. 11/230,989, entitled “MULTI-SITE LEAD/SYSTEM USING A MULTI-POLE CONNECTION AND METHODS THEREFOR.” In one example, at least one of the foregoing parameters are evaluated by way of a logic module 814 of the signal processing circuit 802 and is used in the selection of the one or more electrode configuration used to sense or stimulation the heart 108.

In one example, a stimulation threshold parameter is used in the selection of the one or more electrode configuration for stimulating the heart 108. In varying examples, some or all possible electrode configurations are or may be evaluated to determine which one or more configuration (optimally or acceptably) requires the lowest amount of output energy (i.e., stimulation pulse or shock) be applied to the heart 106 for capturing of the same. In one such example, capturing of the heart 108 is determined by monitoring electrical activity of at least one of the right atrium 220 (FIG. 2), the right ventricle 224 (FIG. 2), the left atrium 222 (FIG. 2), or the left ventricle 226 (FIG. 2) in response to a stimulation pulse or shock of predetermined amplitude. Electrical activity may be determined by using one or more sensor, such as an ultrasound, an accelerometer, or the like, to measure the hemodynamic response to pacing. The presence or absence of such hemodynamic response during an appropriate time period following the stimulation pulse or shock indicates a resulting capture and no capture, respectively.

Advantageously, by providing a system 100 adapted to determine to which one or more electrode configurations require the lowest amount of energy be delivered while still ensuring reliable capture of the heart 108, the life of the IMD 102 may be prolonged, thereby minimizing the risk and expense to the subject 106 (FIG. 1) associated with early explantation and replacement of the IMD. In one example, the system 100 includes an autothreshold determination module 815 adapted to automatically determine whether a stimulation pulse or shock delivered through a first electrode configuration has evoked a desired response from the heart 108, and if not, testing a second, third, . . . , etc. electrode configuration for the desired heart response.

In another example, a stimulation impedance parameter is used in the selection of the one or more electrode configuration for stimulating the heart 108. In varying examples, some or all possible electrode configurations are or may be evaluated to determine which one or more configuration (optimally or acceptably) possess the lowest impedance at an electrode 208/heart tissue 108 interface. Advantageously, by providing a system 100 adapted to determine which one or more electrode configuration possesses the best heart tissue contact, the life of the IMD 102 may be prolonged as result of less battery drain from stimulating the heart.

In another example, a stimulation selection parameter is used in the selection of the one or more electrode configuration for stimulating the heart 108. In varying examples, some or all possible electrode configurations are or may be evaluated to determine which one or more configurations (optimally or acceptably) provides appropriate therapy to one or more chambers of the heart 108 while minimizing phrenic nerve or diaphragmatic stimulation. Advantageously, by providing a system 100 adapted to determine which one or more electrode configurations provides an appropriate balance between pulse or shock stimulation to the heart 108, while minimizing phrenic nerve or diaphragmatic stimulation ensures the subject 106 does not experience undesirable side effects.

In yet another example, a heart chamber configuration parameter is used in the selection of the one or more electrode configuration for stimulating the heart 108. In varying examples, some or all possible electrode configurations are or may be evaluated to determine which one or more configuration (optimally or acceptably) allow for sequential or multi-chamber (e.g., four-chamber) stimulation of the heart for optimum hemodynamic responses. In still another example, a spatial distance parameter is used in the selection of the one or more electrode configuration for stimulating the heart 108.

As illustrated in the example of FIG. 8, the IMD 102 may include the sense/stimulation energy delivery circuit 804 and the sense measurement circuit 806 to sense intrinsic or responsive activity of (e.g., in the form of sense indication signals), and provide stimulation (e.g., pacing, defibrillation, or cardioversion) to, the heart 108, respectively. In one such example, but not by way of limitation, the sense/stimulation energy delivery circuit 804 delivers a pacing pulse stimulation via a lead 104 (FIG. 2) to one or more electrode 208 located in a right ventricle of the heart 108. Such pacing stimuli are usually delivered at a time when the particular heart chamber is in a relaxed, passive state and is being filled with blood. If the delivered pacing stimulus captures the heart, myocardial tissue near the pacing site of the electrode 208 begins to contract, which may be detected by the sense measurement circuit 806. If the delivered pacing stimulus does not capture heart 108 (which may also be detected by the sense measurement circuit806), such tissue does not begin to contract. Similarly, defibrillation or cardioversion stock stimulation may also be applied to the heart 108, with responsive heart activity detected by the sense measurement circuit 806. In addition, the IMD 102 may include the electrode configuration multiplexer 810 to electrically connect electronics of the IMD to the one or more selected electrode configuration.

FIG. 8 illustrates one conceptualization of various circuits, modules, and devices, which are implemented either in hardware or as one or more sequence of steps carried out on a (micro)processor or other controller. Such circuits, modules, and devices are illustrated separately for conceptual clarity; however, it is to be understood that the various circuits, modules, and devices of FIG. 8 need not be separately embodied, but may be combined or otherwise implemented, such as in hardware, software, or firmware. Although not shown in FIG. 8, the IMD 102, such as the signal processing circuit 802, may further include amplification, demodulation, filter, analog-to-digital (A/D) conversion, digital-to-analog (D/A) conversion, and other circuits for extracting and storing information obtained through the system 100.

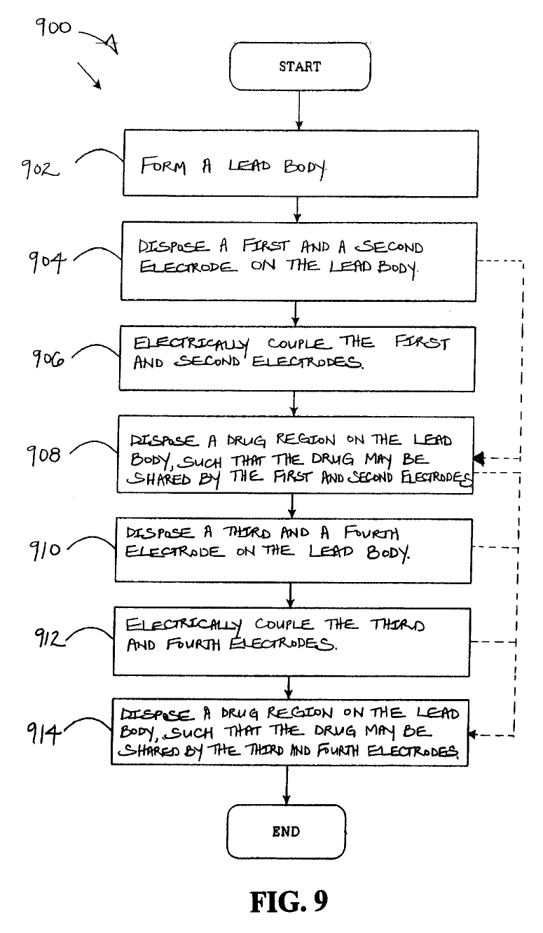

FIG. 9 is a flow chart illustrating a method 900 of manufacturing a lead for use in a system adapted to sense or stimulate a heart, brain, or other desired region of a subject. At 902, a lead body extending from a lead proximal end portion to a lead distal end portion, and having an intermediate portion therebetween, is formed. In one example, forming the lead body includes forming a preformed bias portion at one or both of the lead intermediate or distal end portion. In one such example, the preformed bias portion includes a two-dimensional shape, such as a sinusoidal curve or wave. In another such example, the preformed biased portion includes a three-dimensional shape, such as a helical or other shape that conforms to heart anatomy.

At 904, a first and a second electrode are disposed on the lead body. The first and second electrodes are typically disposed on the lead intermediate or distal end portion. The preformed biased portion, as mentioned in association with 902, is one option for increasing the probability of optimal or acceptable interfacing between the first and second electrodes and tissue or veins of the heart, such as a coronary vein. At 906, the first and second electrodes are optionally electrically coupled. By coupling the first and second electrodes, a larger (effective) surface area is created, thereby increasing the probability of making a satisfactory electrical connection between the electrodes and desired bodily tissue to be sensed or stimulated. At 908, a drug region is disposed on the lead body, such that a drug therein may be shared by the first and second electrodes. In varying examples, the drug region is disposed between the first and second electrodes on the lead body.

At 910, a third and a fourth electrode spaced from the first and second electrodes are optionally disposed on the lead body. At 912, the third and fourth electrodes are optionally electrically coupled. At 914, another drug region is disposed on the lead body, such that a drug therein may be shared by the third and fourth electrodes. Additionally, the method may further include coupling a terminal pin and at least one terminal ring (collectively, one example of a “connection assembly” as referred to herein) are coupled along the lead proximal end portion. The connection assembly is configured to electrically and mechanically couple with a cavity and electrical connections of a medical device, such as an IMD. Further yet, the method may comprise disposing two or more conductors within the lead body, thereby electrically coupling the electrodes and the connection assembly.

The lead constructions discussed herein provide numerous advantages over conventional lead designs including, among other things, a minimization of a drug amount needed (on a per lead basis) and a reduction or elimination of new drug safety and efficacy testing required (as drug dosage is similar to historical data), while still allowing multiple sensing/stimulation vectors and electrode spacing.

It is to be understood that the above description is intended to be illustrative, and not restrictive. For instance, although a majority of the foregoing discusses lead characteristics individually or in specific combinations, any combination of the lead characteristics described herein is within the scope of the present subject matter. In addition, while the above text discusses and figures illustrate, for the most part, implantable leads for use in cardiac situations, the present subject matter is not so limited. Many other embodiments and contexts, such as for non-cardiac nerve and muscle situations (e.g., neurological situations) or for external nerve and muscle situations, will be apparent to those of skill in the art upon reviewing the above description. The scope should, therefore, be determined with reference to the appended claims, along with the full scope of legal equivalents to which such claims are entitled.

What is claimed is:

1. A lead comprising:
a lead body extending from a lead proximal end portion to a lead distal end portion, and having a lead intermediate portion therebetween;
an electrical connector assembly coupled to the lead proximal end portion;
at least a first and a second electrode disposed along the lead body, the electrodes electrically coupled to the connector assembly by way of one or more longitudinally extending conductors; and
a drug region disposed between the first and the second electrodes, the drug region configured to be shared by the electrodes.

2. The lead of claim 1, wherein the drug region extends from a first end to a second end, the first end being between about 0-about 0.250″ from the first electrode and the second end being between about 0-about 0.250″ from the second electrode.

3. The lead of claim 1, further comprising a structural strength of fixation mechanism disposed on the lead body near an edge of the drug region.

4. The lead of claim 1, wherein the drug region comprises a polymeric material mixed with a drug.

5. The lead of claim 4, wherein the drug is dispersed through the polymeric material and a combination thereof is formed into a solid shape couplable with the lead body.

6. The lead of claim 1, wherein the drug region comprises a drug eluting matrix that elutes one or more drugs over time.

7. The lead of claim 6, wherein the drug eluting matrix comprises at least one drug and at least one drawing agent, the drawing agent having the ability to draw bodily fluid into the matrix for modulating a drug delivery rate of the at least one drug to nearby bodily tissue.

8. The lead of claim 1, wherein the lead body comprises a preformed bias portion at one or both of the lead intermediate or the lead distal end portion.

9. The lead of claim 8, wherein the preformed bias portion urges one or more of the first electrode, the second electrode, or the shared drug region against a vessel wall, a septal wall, a heart wall, a pulmonary trunk wall, or a pulmonary artery wall.

10. The lead of claim 8, wherein the preformed bias portion comprises at least one of a helical or sinusoidal shape.

11. A cardiac system including the lead of claim 1, coupled with a medical device via the electrical connector assembly, wherein the medical device comprises an electronics circuit configured to generate one or both of a sense signal or a stimulation signal.

12. The cardiac system of claim 11, wherein the sense or stimulation signal are delivered using one or both of the first or second electrode.

13. The cardiac system of claim 12, wherein the medical device further comprises a processing circuit adapted to select the delivering electrode using, at least in part, one or a combination of a stimulation threshold parameter, a stimulation impedance parameter, a stimulation selection parameter, a heart chamber configuration parameter, or a spatial distance parameter.

14. An implantable lead for use with an implantable medical device, the implantable lead comprising:
a lead body extending from a proximal end to a distal end and having at least one elongated electrical conductor contained therewithin and extending between the proximal end and the distal end;
two or more electrodes disposed on the lead body and electrically coupled with the at least one conductor; and
a drug region positioned directly adjacent the two or more electrodes, the drug region configured to dispense a drug to the electrodes.

15. The implantable lead of claim 14, wherein the drug region is positioned between the two or more electrodes.

16. The implantable lead of claim 15, wherein the drug region includes a fixation mechanism.

17. The implantable lead of claim 14, wherein the two or more electrodes comprises a first, a second, a third, and a fourth electrode; and
wherein a first drug region is positioned between the first and second electrodes and a second drug region is positioned between the third and fourth electrodes.

18. The implantable lead of claim 17, wherein one or both of the first and second electrodes or the third and fourth electrodes are electrically coupled providing an increased effective electrode surface area.

19. The implantable lead of claim 18, wherein the electrical coupling comprises a hard connection between the electrodes or a software connection programmed in an electrically coupled medical device.

20. A method comprising:
forming a lead body encasing a substantial portion of one or more electrical conductors, including forming a lead body extending from a proximal end portion to a distal end portion and having an intermediate portion therebetween;
disposing a first electrode on the lead body near the lead intermediate or distal end portion;
disposing a second electrode on the lead body a selected distance away from the first electrode; and
disposing a drug region on the lead body in a position such that the drug is shared by the first and second electrodes.

21. The method of claim 20, wherein disposing the drug region on the lead body includes disposing the drug region on a portion of the lead body between the first and second electrodes, such that the electrodes straddle the drug region.

22. The method of claim 20, wherein disposing the drug region on the lead body includes spraying, dipping, or painting the drug on the lead body.

23. The method of claim 20, wherein disposing the drug region on the lead body includes impregnating a porous medium with drug on the lead body.

24. The method of claim 20, wherein disposing the drug region on the lead body includes fusing a drug ring to the lead body.

25. The method of claim 20, further comprising electrically coupling the first and second electrodes to provide an increased effective electrode surface area.

26. The method of claim 20, wherein forming the lead body includes forming a bias portion at or near the lead intermediate or distal end portion.

27. The method of claim 20, further comprising disposing a third and a fourth electrode on the lead body, and a drug region therebetween.
